(19) United States
(12) Patent Application Publication
(10) Pub. No.: US 2009/0252704 A1
(43) Pub. Date: Oct. 8, 2009

(54) THERAPIES FOR COGNITION AND LEARNING ENHANCEMENT

(76) Inventors: Allan M. GREEN, Cambridge, MA (US); Robert Bender, Ottawa (CA)

Correspondence Address: LAHIVE & COCKFIELD, LLP;FLOOR 30, SUITE 3000, ONE POST OFFICE SQUARE, BOSTON, MA, 02109, US

(21) Appl. No.: 12/353,407

(22) Filed: Jan. 14, 2009

Related U.S. Application Data
(60) Provisional application No. 61/020,794, filed on Jan. 14, 2008.
(60) Provisional application No. 61/025,945, filed on Feb. 4, 2008.

(51) Int. Cl. A61K 38/20; A61K 31/34; A61K 31/473; A61K 31/425; A61K 31/13; A61K 31/55; A61K 31/27; A61K 31/4353; A61K 38/18

(52) U.S. Cl. 424/85.2; 514/471; 514/297; 514/365; 514/662; 514/217; 514/490; 514/319; 514/12

(57) ABSTRACT

The invention relates to a combination comprising an amount of an NO donor, such as ISDN, and/or an amount of another pharmaceutical agent that enhances neurotransmission or which acts as neuroprotectants such as memantine, clomethiazole and tacrine. These compositions can be used in producing cognition and learning enhancement, whereby the invention also provides for a new method of treatment of Alzheimer's disease and related neurodegenerative disorders.

### RELATED APPLICATIONS

This application claims priority to U.S. Provisional Application No. 61/020,794, Attorney Docket No. SGCI-001-1, filed Jan. 14, 2008, titled “THERAPIES FOR COGNITION AND LEARNING ENHANCEMENT.” This application also claims priority to U.S. Provisional Application No. 61/025,945, Attorney Docket No. SGCI-001-2, filed Feb. 4, 2008, titled “THERAPIES FOR COGNITION AND LEARNING ENHANCEMENT.” The contents of any patents, patent applications, and references cited throughout this specification are hereby incorporated by reference in their entireties.

### BACKGROUND

A number of pathological states, diseases, and disorders are characterized by a profound aberration in the normal function of the central nervous system (CNS). Such conditions include neurodegenerative disorders and diseases such as Parkinson's disease, Huntington's disease, Alzheimer's disease, and epilepsy. These disorders and diseases have had a profound impact in the human populations. For example, Alzheimer's disease accounts for about 70% of all cases of dementia and affects some 2-4 million Americans. As many as 9 million Americans may have Alzheimer's disease by the year 2050. Epidemiological studies have estimated that if Alzheimer's disease could be delayed by 5 years, the incidence and prevalence of this condition would be cut in half. Although much is known about the disease, there are no currently available means of early diagnosis or effective treatment.

Symptoms of neurodegenerative disorders include deterioration of cognition, memory and language.

Accordingly, methods and compositions that can be used for inhibiting neurodegeneration and/or effecting neuroprotection in a subject in need thereof are needed.

### SUMMARY OF THE INVENTION

There remains a need for new treatments and therapies for inhibiting neurodegeneration and/or effecting neuroprotection.

Accordingly, in one aspect, the invention provides a method for inhibiting neurodegeneration, or effecting neuroprotection in a subject in need thereof, said method comprising administering to said subject ISDN, such that said neurodegeneration is inhibited or said neuroprotection is effected. In one embodiment, the ISDN is administered in a low dose. In another embodiment, the low dose does not induce hypotension in a subject. In still another embodiment, the low dose is lower than a dose of ISDN that produces vasodilation in the subject.

In another embodiment of the method of the invention, the ISDN is administered in combination with an additional neuroprotective agent. In still another embodiment, the neuroprotective agent is an acetyl cholinesterase inhibitor, or an NMDA receptor antagonist. In another embodiment, the acetyl cholinesterase inhibitor is tacrine. In yet another embodiment, the NMDA receptor antagonist is memantine. In still another embodiment, the neuroprotective agent is clomethiazole. In still another embodiment, the neuroprotective agent is GT-1061. In another embodiment, the neuroprotective agent is galantamine, rivastigmine, or donepezil. In another embodiment, the ISDN is administered orally, intravenously, buccally, transdermally or subcutaneously. In another embodiment, the method further comprises administering the ISDN in a pharmaceutically acceptable vehicle.

In another aspect, the invention provides a method for inhibiting neurodegeneration, or effecting neuroprotection in a subject in need thereof, said method comprising administering to said subject an effective amount of a therapeutic composition comprising an NO donor, and a neuroprotective agent, such that said neurodegeneration is inhibited or said neuroprotection is effected. In one embodiment, the NO donor is nitroglycerin (GTN), isosorbide 5-mononitrate (ISMN), isosorbide dinitrate (ISDN), pentaerythritol tetranitrate (PETN), erthrityl tetranitrate (ETN), S,S-dinitrosodithiol (SSDD), [N-[2-(nitroxyethyl)]-3-pyridinecarboxamide (nicorandil), sodium nitroprusside (SNP), 4-methyl-5-(2-nitroxyethyl)thiazole HCl or S-nitroso-N-acetylpenicilamine (SNAP). In one embodiment, the NO donor is ISDN, ISMN, 4-methyl-5-(2-nitroxyethyl)thiazole HCl, or GTN.

In another embodiment, the neuroprotective agent is

a) selected from the group consisting of insulin-like growth factor-I (IGF-I), insulin growth-like factor-II (IGF-II), transforming growth factor-β1, activin, growth hormone, nerve growth factor, growth hormone binding protein, IGF-binding proteins (especially IGFBP-), basic fibroblast growth factor, acidic fibroblast growth factor, the hst/Kfgk gene product, fibroblast growth factor 3 (FGF-3), fibroblast growth factor 4 (FGF-4), fibroblast growth factor 6 (FGF-6), keratinocyte growth factor, androgen-induced growth factor, int-2, fibroblast growth factor homologous factor-1 (FHF-1), fibroblast growth factor homologous factor-2 (FHF-2), fibroblast growth factor homologous factor-3 (FHF-3), fibroblast growth factor homologous factor-4 (FHF-4), karatinocyte growth factor 2, glial-activating factor, fibroblast growth factor-10 (FGF-10), fibroblast growth factor-16 (FGF-16), ciliary neurotrophic factor, brain derived growth factor, neurotrophin 3, neurotrophin 4, bone morphogenetic protein 2 (BMP-2), glial-cell line derived neurotrophic factor, activity-dependant neurotrophic factor, cytokine leukemia, inhibiting factor, oncostatin M, interleukin, tumor necrosis factor-α (TNF-α), clomethiazole, kynurenic acid, Semax, FK506 (tacrolimus), L-threo-1-phenyl-2-decanoylamino-3-morpholino-1-propanol, andrenocorticotropin-(4-9 analogue (ORG 2766)) and dizolcipine (MK-801), and selegiline; or

b) the neuroprotective agent is a glutamate antagonist selected from the group consisting of NPS1506, GV1505260, MK-801, and GV150526; or

c) the neuroprotective agent is an alpha-amino-3-hydroxy-5-methyl 4-isoxazole proprionic acid (AMPA) antagonist selected from the group consisting of 2,3-dihydroxy-6-nitro-7-sulfamoylbenzo(f)-quinoxaline (NBQX), LY303070 and LY300164; or

d) the neuroprotective agent is an anti-inflammatory agent directed against mucosal vascular addressing cell adhesion molecular 1 (MAdCAM-1) and/or integrin α4 receptors or anti-MAdCAM-1mAb MECA-367 (ATCC accession no. HB-9478); or

e) said neuroprotective agent is a combination of two or more agents provided in a) through d).

In another embodiment, the neuroprotective agent is an acetyl cholinesterase inhibitor, or an NMDA receptor antagonist. In one embodiment, the acetyl cholinesterase inhibitor is tacrine. In still another embodiment, the NMDA receptor inhibitor is memantine. In yet another embodiment, the neuroprotective agent is clomethiazole. In still another embodiment, the neuroprotective agent is GT-1061.

In one embodiment, the neuroprotective agent is administered at a dose lower than the clinical dose of the neuroprotective agent necessary to inhibit neurodegeneration or effect neuroprotection in a subject. In another embodiment, the neuroprotective agent is administered at a dose of about 0.1 mg/kg to about 10 mg/kg.

In one embodiment, the therapeutic composition of the invention is administered orally, intravenously, buccally, transdermally or subcutaneously. In still another embodiment, the therapeutic combination of the invention is administered with a pharmaceutically acceptable vehicle. In another embodiment, administering the therapeutic composition of the invention to a subject modulates brain NO levels.

In another aspect, the invention provides a method for inhibiting neurodegeneration or effecting neuroprotection in a subject in need thereof, said method comprising administering to said subject an effective amount of a therapeutic composition comprising ISDN and clomethiazole, ISDN and tacrine, or ISDN and memantine.

In another embodiment, the neurodegeneration or said neuroprotection of the methods of the invention are associated with a condition selected from the group consisting of stroke, Parkinson's disease, Alzheimer's disease, Huntington's disease, multiple sclerosis, amyotrophic lateral sclerosis, AIDS-induced dementia, epilepsy, alcoholism, alcohol withdrawal, drug-induced seizure, viral/bacterial/fever-induced seizure, trauma to the head, hypoglycemia, hypoxia, myocardial infarction, cerebral vascular occlusion, cerebral vascular hemorrhage, hemorrhage, an environmental excitotoxin, dementia, trauma, drug-induced brain damage, and aging. In another embodiment, the neurodegeneration or said neuroprotection is associated with Alzheimer's disease. In another embodiment, the neurodegeneration or said neuroprotection is associated with dementia.

In another embodiment, the invention provides an NO donor and a neuroprotective agent. In another aspect, the invention provides a pharmaceutical composition comprising ISDN and clomethiazole. In another aspect, the invention provides a pharmaceutical composition comprising ISDN and memantine. In still another aspect, the invention provides a pharmaceutical composition comprising ISDN and tacrine.

In another aspect, the invention provides a method for the enhancement of learning and cognition and for the treatment of disorders characterized by learning and cognition deficits in a subject in need thereof, comprising administering to said individual an effective amount of an NO donor, in combination with an effective amount of one or more agents which enhance neurotransmission or which act as neuroprotectants, wherein said combination has an effect on learning and cognition. In one embodiment, the NO donor is ISDN. In another embodiment, the NO donor provides NO to the CNS but does not cause clinically significant hypotension. In another embodiment, the combination of the NO donor and agent is greater than additive.

In another aspect, the invention provides a method for the treatment of cognition and learning deficits in a subject in need thereof, comprising administering to said individual an effective amount of an NO donor that provides NO to the CNS but does not cause clinically significant hypotension, in combination with an effective amount of a neurotransmitter or a neuroprotective agent, wherein said combination has the effect of enhancing learning and cognition which is persistent over 14 days or longer at a greater level than the effect on learning and cognition induced by the neurotransmitter or the neuroprotective agent alone. In another embodiment, the NO donor is ISDN. In still another embodiment, the neurotransmitter or the neuroprotective agent is selected from the group consisting of memantine, tacrine, and clomethiazole. In another embodiment, the neurotransmitter or the neuroprotective agent is selected from the group consisting of galantamine, rivastigmine, and donepezil. In another embodiment, the disorders characterized by learning and cognition deficits are related to Alzheimer's disease

In another aspect, the invention provides an NO donor, which provides NO to the CNS but does not cause clinically significant hypotension, packaged as a separate medication and intended for administration in combination with one or more neurotransmitter enhancers or neuroprotectants. In one embodiment, the NO donor is ISDN.

In another aspect, the invention provides an NO donor, which provides NO to the CNS but does not cause clinically significant hypotension, compounded with one or more neurotransmitter or neuroprotective agents as a combination drug for oral, buccal, transdermal, intrathecal or parenteral administration. In one embodiment, the NO donor is ISDN.

In another aspect, the invention provides a method of treating Alzheimer's disease, using a pharmaceutical composition comprising ISDN and clomethiazole. In another aspect, the invention provides a method of treating Alzheimer's disease, using a pharmaceutical composition comprising ISDN and GT-1061.

In one embodiment of the methods of the invention, the subject is human.

In another embodiment, the invention provides a pharmaceutical composition comprising ISDN and galantamine. In another embodiment, the invention provides a pharmaceutical composition comprising ISDN and rivastigmine. In another embodiment, the invention provides a pharmaceutical composition comprising ISDN and donepezil. In yet another embodiment, the invention provides a pharmaceutical composition comprising GTN and clomethiazole. In another embodiment, the invention provides a pharmaceutical composition comprising GTN and memantine. In another embodiment, the invention provides a pharmaceutical composition comprising GTN and tacrine. In still another embodiment, the invention provides a pharmaceutical composition comprising GTN and galantamine. In another embodiment, the invention provides a pharmaceutical composition comprising GTN and rivastigmine. In still another embodiment, the invention provides a pharmaceutical composition comprising GTN and donepezil. In another embodiment, the invention provides a pharmaceutical composition comprising ISDN and PRX-03140. In still another embodiment, the invention provides a pharmaceutical composition comprising GTN and PRX-03140. Any of these pharmaceutical compositions can be delivered to a subject in need thereof in conjunction with a transdermal patch. These pharmaceutical compositions can also be administered in a controlled-release fashion.

### BRIEF DESCRIPTION OF THE DRAWINGS

FIG. 1, FIG. 2 and FIG. 3 demonstrate the cognition deficit reversal by GT-1061 in drinking water as discussed in the exemplification section.

FIG. 4 shows a comparison of reversal of cognition deficits by GT-1061, clomethiazole, and ISDN.

FIG. 5 shows the efficacy of a composition comprising a combination of ISDN and clomethiazole in reversal of cognition deficit.

### DETAILED DESCRIPTION OF THE INVENTION

This invention is directed toward compositions, as well as pharmaceutical compositions, for use in inhibiting neurodegeneration, and/or effecting neuroprotection in a subject in need thereof. The invention relates to isosorbide dinitrate (ISDN) for the enhancement of neurotransmission or as a neuroprotectant, as well as a combination comprising a nitrate (NO) donor such as ISDN, and one or more agents that enhance neurotransmission or which act as neuroprotectants. The NO donor may be supplied in a package containing dosage units comprising said NO donor with or without the neurotransmission enhancer or neuroprotectant. The invention also relates to a method of treatment of cognition and learning impairment and related disorders.

### DEFINITIONS

The term “treat,” “treated,” “treating” or “treatment” includes the diminishment or alleviation of at least one symptom associated or caused by the state, disorder or disease being treated. In certain embodiments, the treatment comprises the induction of neurodegeneration, followed by the activation of the composition of the invention, which would in turn diminish or alleviate at least one symptom associated or caused by the neurodegeneration being treated. For example, treatment can be diminishment of one or several symptoms of a disorder or complete eradication of a disorder.

The term “use” includes any one or more of the following embodiments of the invention, respectively: the use in the treatment of neurodegeneration and/or effecting neuroprotection; the use for the manufacture of pharmaceutical compositions for use in the treatment of these diseases, e.g., in the manufacture of a medicament; methods of use of compositions of the invention in the treatment of these diseases; pharmaceutical preparations having compositions of the invention for the treatment of these diseases; and compositions of the invention for use in the treatment of these diseases; as appropriate and expedient, if not stated otherwise. In particular, diseases to be treated and are thus preferred for use of a composition of the present invention are neurodegeneration and/or effecting neuroprotection.

The term “subject” is intended to include organisms, e.g., prokaryotes and eukaryotes, which are capable of suffering from or afflicted with a neurodegeneration and/or in need of neuroprotection. Examples of subjects include mammals, e.g., humans, dogs, cows, horses, pigs, sheep, goats, cats, mice, rabbits, rats, and transgenic non-human animals. In certain embodiments, the subject is a human, e.g., a human suffering from, at risk of suffering from, or potentially capable of suffering from neurodegeneration and/or in need of neuroprotection. In another embodiment, the subject is a cell.

As used herein, “therapeutically effective amount” refers to an amount of a first compound (e.g., ISDN) and/or a second compound (e.g., tacrine, memantine or clomethiazole), as used herein, sufficient to elicit a desired biological response. In the methods described herein, a desired biological response is a reduction (complete or partial) of at least one symptom associated with the disorder being treated and/or improved efficacy. As with any treatment, particularly treatment of a multi-symptom disorder, it is advantageous to treat as many disorder-related symptoms as the patient experiences. The phrase “therapeutically effective amount” encompasses amounts of a first compound and optionally a second compound, as described herein, wherein the combination of the first and at least one second compound results in treatment of neurodegeneration and/or effecting neuroprotection. Any amounts of a first compound and optionally a second compound as described herein can be used in the prevention, treatment, and optionally management of a disorder, as described herein

As used herein, the term “pharmaceutically acceptable excipient” or “excipient” includes compounds that are compatible with the other ingredients in a pharmaceutical formulation and not injurious to the subject when administered in therapeutically effective amounts.

As used herein, the term “pharmaceutically acceptable salt” includes salts that are physiologically tolerated by a subject. Such salts are typically prepared from an inorganic and/or organic acid. Examples of suitable inorganic acids include, but are not limited to, hydrochloric, hydrobromic, hydroiodic, nitric, sulfuric, and phosphoric acid. Organic acids may be aliphatic, aromatic, carboxylic, and/or sulfonic acids. Suitable organic acids include, but are not limited to, formic, acetic, propionic, succinic, camphorsulfonic, citric, fumaric, gluconic, lactic, malic, mucic, tartaric, para-toluenesulfonic, glycolic, glucuronic, maleic, furoic, glutamic, benzoic, anthranilic, salicylic, phenylacetic, mandelic, pamoic, methanesulfonic, ethanesulfonic, pantothenic, benzenesulfonic (besylate), stearic, sulfanilic, alginic, galacturonic, and the like.

The phrase “pharmaceutically effective amount” as used herein indicates an amount necessary to administer to a host, or to a cell, issue, or organ of a host, to achieve a therapeutic result, especially the regulating, modulating, or inhibiting a neurodegenerative disorder, or effecting cognition.

The term “combination” refers to any presentation form in which the intention for combined use of NO donors, such as ISDN, and one or more cognition enhancers or neuroprotectants can be recognized. Such combinations of NO donors and neurotransmission enhancers and/or neuroprotectant agents may in this description also be referred to as combinations according to the invention. It will be appreciated that the compounds of the combination may be administered concomitantly, either in the same or different pharmaceutical formulation, or sequentially. If there is sequential administration, the delay in administering the second (or additional) active ingredient should not be such as to lose the benefit of the efficacious effect of the combination of the active ingredients. A minimum requirement for a combination according to this description is that the combination should be intended for combined use with the benefit of the efficacious effect of the combination of the active ingredients. The intended use of a combination can be inferred by facilities, provisions, adaptations and/or other means to help using the combination according to the invention. For example, a combination can be made suitable by adding instructions or aids or even determinants for the combined use. Determinants for the combined use can, for example, reside in the properties of a dispenser of dosage units of the active ingredients of the combination. The active ingredients can thus be in separate dosage units, but still the combination can have a determinant inducing the use of the dosage units of the combination in a predetermined sequence and/or at pre-determined times by the properties of the dispenser. A preferred determinant for combined use is the formulation of both the active components of the combination in one pharmaceutical composition. Thus, according to one aspect, the present invention provides a pharmaceutical composition, comprising ISDN, or a pharmaceutically acceptable salt or solvate thereof, and a neurotransmitter enhancer such as tacrine or memantine, or a neuroprotectant such as clomethiazole, or pharmaceutically acceptable salts or solvates thereof.

### Compositions of the Invention

It has long been known that nitrates (NO) generated in the CNS during trauma or stroke may be detrimental to the maintenance of normal neuronal functioning. It is therefore surprising that NO donors, such as ISDN, act as NO donors in chronic use and have been shown to enhance learning and cognition in animal models of chronic learning and cognition impairment. Moreover, it is surprising that a synergistic effect is found with NO donors, such as ISDN, and neurotransmitter enhancers and neuroprotectants such as, but not limited to, tacrine, memantine and clomethiazole.

This invention provides for a combination comprising an amount of ISDN, or a pharmaceutically acceptable salt or solvate thereof, wherein the ISDN can be administered without causing clinically unacceptable hypotension, for use as a neuroprotectant, and/or to enhance neurotransmission in a subject in need thereof.

This invention also provides an NO donor such as ISDN, which can be administered without causing clinically unacceptable hypotension, and an amount of one or more agents which enhance neurotransmission or which act as neuroprotectants, or a pharmaceutically acceptable salt or solvate thereof, optionally in association with one or more pharmaceutically acceptable carriers, whereby the amount of the NO donor and the amount of the neuroprotectants or neurotransmitter enhancers are such that the effect of the combination is more favorable than the added effects of the amounts of each drug separately. The drugs may be formulated separately for administration as a combination therapy. Alternatively, the drugs may be formulated together as a single combination drug product.

For purposes of the present invention, by the term “NO donor,” or “nitric oxide mimetic” it is meant nitric oxide, or a functional equivalent thereof; any compound that mimics the effects of nitric oxide, generates or releases nitric oxide through biotransformation, generates nitric oxide spontaneously, or spontaneously releases nitric oxide; any compound that in any other manner generates nitric oxide or a nitric oxide-like moiety or activates other stages of the NO pathway; or any compound that enables or facilitates NO utilization by the cell, when administered to an animal can also be referred to as “NO donors,” “NO prodrugs,” “NO producing agents,” “NO delivering compounds,” “NO generating agents,” or “NO providers.” Examples of such compounds include, but are not limited to: organic nitrates such as nitroglycerin (GTN), isosorbide 5-mononitrate (ISMN), isosorbide dinitrate (ISDN), pentaerythritol tetranitrate (PETN), erthrityl tetranitrate (ETN), 4-methyl-5-(2-nitroxyethyl)thiazole HCl, amino acid derivatives such as S-nitrosoglutathione (SNOG); and other compounds that generate or release NO under physiologic conditions such as S,S-dinitrosodithiol (SSDD), [N-[2-(nitroxyethyl)]-3-pyridinecarboxamide (nicorandil), sodium nitroprusside (SNP), S-nitroso-N-acetylpenicilamine (SNAP), 3-morpholino-sydnonimine(SIN-1), molsidomine, DEA-NONOate (2-(N,N-diethylamino)-diazenolate-2-oxide), and spermine NONOate (N-[4-[1-(3-aminopropyl)-2-hydroxy-2-nitrosohydrazino]butyl-1,3-propanediamine). The organic nitrates GTN, ISMN, ISDN, ETN, and PETN, as well as nicorandil (commonly known as a potassium channel opener) are commercially available in pharmaceutical dosage forms. SIN-1, SNAP, S-nitrosoglutathione, L-NIO, spermine NONOate, and DEA-NONOate are commercially available from Biotium, Inc. Richmond, Calif. As used herein, the term “NO donor” is also intended to mean any compound that acts as a nitric oxide pathway mimetic, that has nitric oxide-like activity, or that mimics the effect of nitric oxide. Such compounds may not necessarily release, generate or provide nitric oxide, but they have a similar effect to nitric oxide on a pathway that is affected by nitric oxide. For example, nitric oxide has both cyclic GMP-dependent and cyclic GMP-independent effects. Nitric oxide is known to activate the soluble form of guanylyl cyclase thereby increasing intracellular levels of the second messenger cyclic GMP and other interactions with other intracellular second messengers such as cyclic AMP. As such, compounds that directly activate either particulate or soluble guanylyl cyclase such as natriuretic peptides (ANP, BNP, and CNP), 3-(5′-hydroxymethyl-2′furyl)-1-benzyl indazole (YC-1) and 8-(4-chlorophenylthio)guanosine 3′,5′-cyclic monophosphate (8-PCPT-cGMP), are also examples of NO-mimetics. In some embodiments of the present invention, however, it is preferred that the NO-mimetic not encompass a compound that directly activates either particulate or soluble guanylyl cyclase. Nitric oxide mimetic activity encompasses those signal transduction processes or pathways that comprise at least one NO mimetic-binding effector molecule, such as for example, guanylyl cyclase and other heme containing proteins. Example of agents that function as NO mimetics by enabling or facilitating NO utilization by the cell are compounds that inhibit phosphodiesterase activity and/or expression, such as phosphodiesterase inhibitors. The present invention also includes the NO donors disclosed in U.S. Pat. No. 6,946,484, which is incorporated herein by reference in its entirety.

In a preferred embodiment of the present invention, the NO donor is ISDN.

As used herein, the term “neuroprotective agent” refers to an agent that prevents or slows the progression of neuronal degeneration and/or prevents neuronal cell death.

The term “neurotransmitter enhancer” means a compound whose biochemical effect results in increased neuronal and/or neurotransmitter function.

In one embodiment, the neuroprotective agent is

a) selected from the group consisting of insulin-like growth factor-I (IGF-I), insulin growth-like factor-II (IGF-II), transforming growth factor-β1, activin, growth hormone, nerve growth factor, growth hormone binding protein, IGF-binding proteins (especially IGFBP-), basic fibroblast growth factor, acidic fibroblast growth factor, the hst/Kfgk gene product, fibroblast growth factor 3 (FGF-3), fibroblast growth factor 4 (FGF-4), fibroblast growth factor 6 (FGF-6), keratinocyte growth factor, androgen-induced growth factor, int-2, fibroblast growth factor homologous factor-1 (FHF-1), fibroblast growth factor homologous factor-2 (FHF-2), fibroblast growth factor homologous factor-3 (FHF-3), fibroblast growth factor homologous factor-4 (FHF-4), karatinocyte growth factor 2, glial-activating factor, fibroblast growth factor-10 (FGF-10), fibroblast growth factor-16 (FGF-16), ciliary neurotrophic factor, brain derived growth factor, neurotrophin 3, neurotrophin 4, bone morphogenetic protein 2 (BMP-2), glial-cell line derived neurotrophic factor, activity-dependant neurotrophic factor, cytokine leukemia, inhibiting factor, oncostatin M, interleukin, tumor necrosis factor-α (TNF-α), clomethiazole, kynurenic acid, Semax, FK506 (tacrolimus), L-threo-1-pheyl-2-decanoylamino-3-morpholino-1-propanol, andrenocorticotropin-(4-9 analogue (ORG 2766)) and dizolcipine (MK-801), and selegiline; or

b) the neuroprotective agent is a glutamate antagonist selected from the group consisting of NPS1506, GV1505260, MK-801, and GV150526; or

c) the neuroprotective agent is an alpha-amino-3-hydroxy-5-methyl 4-isoxazole proprionic acid (AMPA) antagonist selected from the group consisting of 2,3-dihydroxy-6-nitro-7-sulfamoylbenzo(f)-quinoxaline (NBQX), LY303070 and LY300164; or

d) the neuroprotective agent is an anti-inflammatory agent directed against mucosal vascular addressing cell adhesion molecular 1 (MAdCAM-1) and/or integrin α4 receptors or anti-MAdCAM-1mAb MECA-367 (ATCC accession no. HB-9478); or

e) said neuroprotective agent is a combination of two or more agents provided in a) through d).

In a particular embodiment, the neurotransmitter enhancers or neuroprotectants to be used with the NO donors of the invention are memantine, tacrine or clomethiazole.

Thus, NO donors and neurotransmitter enhancers or neuroprotectants such as memantine, tacrine or clomethiazole have a synergistic interaction when used in the treatment of Alzheimer's disease and other neurodegenerative disorders. As a consequence, the combined use of NO donors and neurotransmitter enhancers or neuroprotectants has better effects in more patients in comparison to each drug alone. The better effect may be manifest as fewer side effects or a more persistent learning or cognition enhancement effect or an improved level of functioning in individual patients.

The combination of compounds described herein can either result in synergistic increase in anti-neurodegeneration activity and/or effecting of neuroprotection, or such an increase can be additive. Compositions described herein can include lower dosages of each compound in a composition, thereby avoiding adverse interactions between compounds and/or harmful side effects, such as ones which have been reported for similar compounds. Furthermore, normal amounts of each compound when given in combination could provide for greater efficacy in subjects who are either unresponsive or minimally responsive to each compound when used alone.

A synergistic effect can be calculated, for example, using suitable methods such as the Sigmoid-Emax equation (Holford, N. H. G. and Scheiner, L. B., Clin. Pharmacokinet. 6: 429-453 (1981)), the equation of Loewe additivity (Loewe, S, and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied to experimental data to generate a corresponding graph to aid in assessing the effects of the drug combination. The corresponding graphs associated with the equations referred to above are the concentration-effect curve, isobologram curve and combination index curve, respectively.

As discussed above, an advantage of the compositions described herein is the ability to use less of each compound than is needed when each is administered alone. Another advantage is that greater efficacy may be achieved in subjects who are either unresponsive or minimally responsive to each compound when used alone in normal amounts by giving the agents in combination. As such, undesirable side effects associated with the compounds are reduced (partially or completely) and/or improved efficacy may be achieved. A reduction in side effects with or without improved efficacy can result in increased patient compliance over current treatments.

In one aspect, the invention includes a low-dose of an NO donor such as ISDN. In one embodiment, the low dose does not induce hypotension in a subject. In another embodiment, the low dose is lower than a dose of an NO donor that produces vasodilation in a subject.

### Methods of Treatment

This invention provides compounds, combinations of compounds, methods and pharmaceutical compositions that are useful in the treatment of neurological disorders requiring mitigation of neurodegeneration, neuroprotection and/or cognition enhancement. Methods of the invention involve administering to a subject in need thereof a therapeutic compound which provides neuroprotection or cognition enhancement. Accordingly, the compositions and methods of the invention are useful for effecting neuroprotection or cognition enhancement in disorders in which neurotoxic damage occurs.

The methods of the invention can be used therapeutically to treat conditions including, but not limited to: stroke; Parkinson's disease; Alzheimer's disease; Huntington's disease; multiple sclerosis; amylotrophic lateral sclerosis; AIDS-induced dementia; epilepsy; alcoholism; alcohol withdrawal; drug-induced seizures; viral/bacterial/fever-induced seizures; trauma to the head; hypoglycemia; hypoxia; myocardial infarction; cerebral vascular occlusion; cerebral vascular hemorrhage; hemorrhage; environmental excitotoxins; dementias of all type, trauma, drug-induced brain damage, and aging or can be used prophylactically in a subject susceptible or predisposed to these conditions. In certain embodiments, a therapeutic compound used in the method of the invention preferably can interact with GCase effecting neuroprotection and/or cognition enhancement. In other embodiments, a therapeutic compound used in the method of the invention preferably can modulate glutamate and/or non-glutamate neuroreceptor interactions effecting neuroprotection and/or cognition enhancement.

“Mitigating” or “treating” neurodegeneration as used herein involves effecting neuroprotection, inhibiting or preventing neurodegeneration, and/or ameliorating the manifestations or impact of neurodegeneration. Such amelioration includes effecting cognition enhancement, as is quantified by tests known in the art. “Modulating” a biological process as used herein (for example, modulating the activity of the non-glutamate neuroreceptors), encompasses both increasing (positively modulating) and decreasing (negatively modulating) such activity, and thus inhibition, potentiation, agonism, and antagonism of the biological process.

The therapies of the instant invention also can be used to treat the diseases and disorders disclosed in U.S. Pat. No. 6,916,835, which is incorporated herein by reference in its entirety.

### Pharmaceutical Compositions

While it is possible for the active ingredients of the combination to be administered as the raw chemical it is preferable to present them as a pharmaceutical composition, also referred to in this context as pharmaceutical formulation. Suitable compositions include those suitable for oral, rectal, nasal, topical (including transdermal, buccal and sublingual), vaginal or parenteral (including subcutaneous, intramuscular, intravenous and intradermal) administration. Pharmaceutical compositions in embodiments of the present invention comprise ISDN, or a combination of an NO donor such as ISDN, and a neurotransmitter/neuroprotectant, together with one or more pharmaceutically acceptable carriers or excipients and optionally other therapeutic agents.

The language “effective amount” of the composition is that amount necessary or sufficient to treat neurodegeneration and/or effect cognition, e.g. prevent the various morphological and somatic symptoms of a disease or condition described herein. In an example, an effective amount of the composition of the invention is the amount sufficient to treat neurodegeneration and/or effect cognition in a subject. The effective amount can vary depending on such factors as the size and weight of the subject, the type of illness, or the particular composition of the invention. For example, the choice of the composition of the invention can affect what constitutes an “effective amount.” One of ordinary skill in the art would be able to study the factors contained herein and make the determination regarding the effective amount of the compositions of the invention without undue experimentation.

The regimen of administration can affect what constitutes an effective amount. The composition of the invention can be administered to the subject either prior to or after the onset of a disease or disorder. Further, several divided dosages, as well as staggered dosages, can be administered daily or sequentially, or the dose can be continuously infused, or can be a bolus injection. Further, the dosages of the composition(s) of the invention can be proportionally increased or decreased as indicated by the exigencies of the therapeutic or prophylactic situation.

Compositions of the invention (e.g., a composition comprising ISDN and tacrine, memantine, or clomethiazole) may be used in the treatment of states, disorders or diseases as described herein, or for the manufacture of pharmaceutical compositions for use in the treatment of these diseases. Methods of use of compositions of the present invention in the treatment of these diseases, or pharmaceutical preparations having compositions of the present invention for the treatment of these diseases.

The language “pharmaceutical composition” includes preparations suitable for administration to mammals, e.g., humans. When the compositions of the present invention are administered as pharmaceuticals to mammals, e.g., humans, they can be given per se or as a pharmaceutical composition containing, for example, 0.1 to 99.5% (more preferably, 0.5 to 90%) of active ingredient in combination with a pharmaceutically acceptable carrier.

The phrase “pharmaceutically acceptable carrier” is art recognized and includes a pharmaceutically acceptable material, composition or vehicle, suitable for administering compositions of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical, buccal, sublingual, rectal, vaginal and/or parenteral administration. The formulations may conveniently be presented in unit dosage form and may be prepared by any methods well known in the art of pharmacy. The amount of active ingredient that can be combined with a carrier material to produce a single dosage form will generally be that amount of the composition that produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a composition of the present invention with the carrier and, optionally, one or more accessory ingredients. In general, the formulations are prepared by uniformly and intimately bringing into association a composition of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a composition of the present invention as an active ingredient. A composition of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered composition moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the compositions of the invention include pharmaceutically acceptable emulsions, microemulsions, solutions, suspensions, syrups and elixirs. In addition to the active ingredient, the liquid dosage forms may contain inert diluent commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compositions, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compositions of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active composition.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a composition of this invention include powders, sprays, ointments, pastes, creams, lotions, gels, solutions, patches and inhalants. The active composition may be mixed under sterile conditions with a pharmaceutically acceptable carrier, and with any preservatives, buffers, or propellants that may be required.

The ointments, pastes, creams and gels may contain, in addition to an active composition of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a composition of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a composition of the present invention to the body. Such dosage forms can be made by dissolving or dispersing the composition in the proper medium. Absorption enhancers can also be used to increase the flux of the composition across the skin. The rate of such flux can be controlled by either providing a rate controlling membrane or dispersing the active composition in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compositions of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compositions in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

The preparations of the present invention may be given orally, parenterally, topically, or rectally. They are of course given by forms suitable for each administration route. For example, they are administered in tablets or capsule form, by injection, inhalation, eye lotion, ointment, suppository, etc., administration by injection, infusion or inhalation; topical by lotion or ointment; and rectal by suppositories. Oral and/or IV administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracistemally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound that is the lowest dose effective to produce a therapeutic effect. Such an effective dose will generally depend upon the factors described above. Generally, intravenous and subcutaneous doses of the compounds of this invention for a patient, when used for the indicated analgesic effect, will range from about 0.0001 to about 100 mg per kilogram of body weight per day, more preferably from about 0.01 to about 50 mg per kg per day, and still more preferably from about 1.0 to about 100 mg per kg per day. An effective amount is that amount treats neurodegeneration and/or effects cognition.

If desired, the effective daily dose of the active compound may be administered as two, three, four, five, six or more sub-doses administered separately at appropriate intervals throughout the day, optionally, in unit dosage forms.

While it is possible for a compound of the present invention to be administered alone, it is preferable to administer the compound as a pharmaceutical composition.

### Kits

Advantageously, the present invention also provides kits for use by a consumer for treating disease. The kits comprise a) a pharmaceutical composition comprising the NO donor (e.g., ISDN) and optionally a neuroprotective agent, and a pharmaceutically acceptable carrier, vehicle or diluent; and, optionally, b) instructions describing a method of using the pharmaceutical composition for treating the specific disease. The instructions may also indicate that the kit is for treating disease while substantially reducing the concomitant liability of adverse effects associated with antibiotic administration.

A “kit” as used in the instant application includes a container for containing the separate unit dosage forms such as a divided bottle or a divided foil packet. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle which is in turn contained within a box.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It maybe desirable to provide a written memory aid, where the written memory aid is of the type containing information and/or instructions for the physician, pharmacist or subject, e.g., in the form of numbers next to the tablets or capsules whereby the numbers correspond with the days of the regimen which the tablets or capsules so specified should be ingested or a card which contains the same type of information. Another example of such a memory aid is a calendar printed on the card e.g., as follows “First Week, Monday, Tuesday,” . . . etc. . . . “Second Week, Monday, Tuesday, . . . ” etc. Other variations of memory aids will be readily apparent. A “daily dose” can be a single tablet or capsule or several tablets or capsules to be taken on a given day.

Another specific embodiment of a kit is a dispenser designed to dispense the daily doses one at a time. Preferably, the dispenser is equipped with a memory-aid, so as to further facilitate compliance with the regimen. An example of such a memory-aid is a mechanical counter, which indicates the number of daily doses that, has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

More commonly, pharmaceutical formulations are prescribed to the patient in “patient packs” containing a number dosing units or other means for administration of metered unit doses for use during a distinct treatment period in a single package, usually a blister pack. Patient packs have an advantage over traditional prescriptions, where a pharmacist divides a patient's supply of a pharmaceutical from a bulk supply, in that the patient always has access to the package insert contained in the patient pack, normally missing in traditional prescriptions. The inclusion of a package insert has been shown to improve patient compliance with the physician's instructions. Thus, the invention further includes a pharmaceutical formulation, such as ISDN, or a combination of an NO donor such as ISDN, and a neurotransmitter/neuroprotectant as herein before described, in combination with packaging material suitable for said formulations. In such a patient pack the intended use of a formulation for the combination treatment of neurodegeneration or related disorders can be inferred by instructions, facilities, provisions, adaptations and/or other means to help using the formulation most suitably for the treatment. Such measures make a patient pack specifically suitable for and adapted for use for treatment with the combination of the present invention.

Specifically, a further embodiment includes a package containing separate dosage units, one or more of which containing ISDN or another acceptable nitrate (NO) donor, or a pharmaceutically acceptable salt thereof, and one or more of an acceptable neurotransmitter enhancer or neuroprotection agent such as memantine, tacrine or clomethiazole or a pharmaceutically acceptable salt thereof. A package contains enough tablets, capsules or the like to treat a patient for a pre-determined period of time, for instance for 2 weeks, 1 month or 3 months.

### Exemplification of the Invention

The invention is further illustrated by the following examples, which could be used to examine the gated ion channel modulating activity of the compounds of the invention. The example should not be construed as further limiting. The animal models used throughout the Examples are accepted animal models and the demonstration of efficacy in these animal models is predictive of efficacy in humans.

### STPA Study of GT-1061, ISDN, and CMZ

### Purpose

1. To examine the ability of GT-1061 (4-methyl-5-(2-nitroxyethyl)thiazole HCl), clomethiazole (CMZ), and isosorbide dinitrate (ISDN), and combinations thereof to improve cognitive performance in an animal model of dementia relevant to Alzheimer's disease (AD): step through passive avoidance (STPA) in scopolamine treated mice.

2. To examine the effective biological half life in this model after bolus i.p. dosing.

### Methodology

Male C57BL/6 mice weighing 20-25 g (6-8 weeks of age) were maintained in a controlled environment (housed at 5 per cage; at 22-25 C.°; 12/12 light/dark cycle) and allowed to acclimate 2 weeks before initiation of the STPA task. The STPA apparatus consists of a box with two compartments light and dark; each of 20×9.5×16.5 cm³ (Ugo Basile Rat Passive Avoidance Apparatus, customized for mice with chamber inserts). Mice were transported to the testing facility in covered cages and whilst in training or testing phases were housed at 1 or 2 per cage.

STPA Habituation phase: Mice are placed in the light compartment and latency to enter the dark compartment is recorded. The criteria of success is entrance to the dark compartment within 30 sec twice in three trails or three times in five trials; showing the normal aversion of mice to a highly lit space.

STPA Training phase: In acute drug testing, 2 hrs after habituation is complete; 30 min (t=−30) and 20 min (t=−20) prior to training, scopolamine (or saline) and test drug (or vehicle), respectively, are administered. In the drinking water protocol, at t=−30 prior to training, scopolamine (or saline) is administered. Mice are placed into the light compartment at time zero. Upon entry into the dark compartment, an automatic door closes and 2.0 sec shock (0.5 mA) is delivered through the floor grating. This process is continued until the animal does not voluntarily enter the dark side within 300 sec. The number of trials to reach criterion is recorded for assessment of secondary biomarkers, such as analgesia. No consistent differences to reach criterion were noted (FIG. 3).

STPA Testing phase: At 24 h and 48 h post training, animals are tested for memory retention without any drug administration. The animals are again placed in the light compartment. Latency to enter the dark side is recorded as a measure of memory consolidation, retention and retrieval.

Drugs: All drugs dissolved in water or 10-25% DMSO (DMSO has no effect on the retention performance of mice in the STPA, but does cause irritation at the higher volumes required for assessment of sedative activity). Scopolamine (1 mg/kg; 1 mg/4 ml) is administered ip. Test compound (1 mg/kg, 1 mg/Kg) administered ip.

Dosing—Behavioral Studies: Drugs and vehicle were administered either i.p. for acute studies (1 mg/kg, or as indicated in the figures, in saline), otherwise in drinking water (20 mg/kg/day).

Drinking water dosing: Rodents drink continuously during the active dark cycle and intermittently during the resting light cycle. Delivery of drug in drinking water mimics a biphasic extended release formulation that delivers drug continuously during waking hours, but at much lower levels during sleep.

1. Drinking water consumption was monitored over 3 days for 3 cages of animals (5 per cage). Consumption was observed to be highly consistent over days and cages: 24.7 ml (4.9 ml/mouse).

2. For a group of 10 animals, at the beginning of every week make up a solution GT-1061 (35 mg) dissolved in aqueous sodium citrate (trisodium salt dihydrate [Sigma-Aldrich cat# S1804 CAS# 6132-04-3] 17 mg made up to 350 mL with water). This solution can be stored in a 2-4 C fridge for 1 week. At the end of the week, any remaining solution should be transferred to a plastic container and frozen and marked with the date for subsequent assay of drug concentration.

3. For administration beyond 24 h, drug-containing drinking water should be replenished at the same time every 24 h: (a) the remaining drinking water should be measured in a simple measuring cylinder and the volume recorded by animal number and date; (b) the remaining water and sufficient fresh solution should be added to the drinking water container on the basis of 5 mL of drug solution per animal per day. A cage with 3 animals requires 15 mL per day; 4 animals requires 20 mL per day, etc.

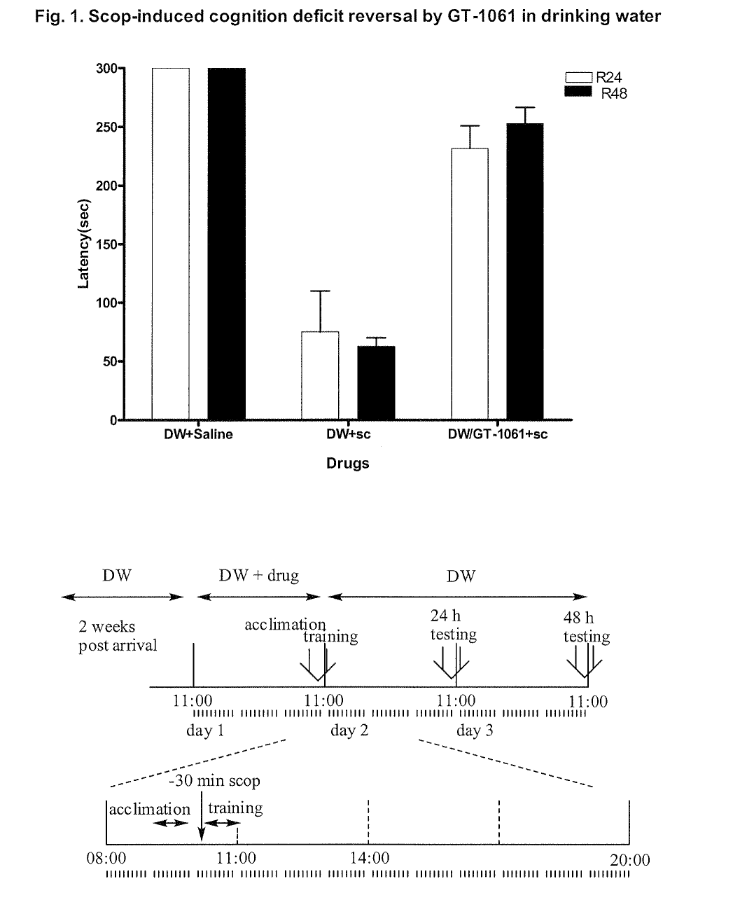

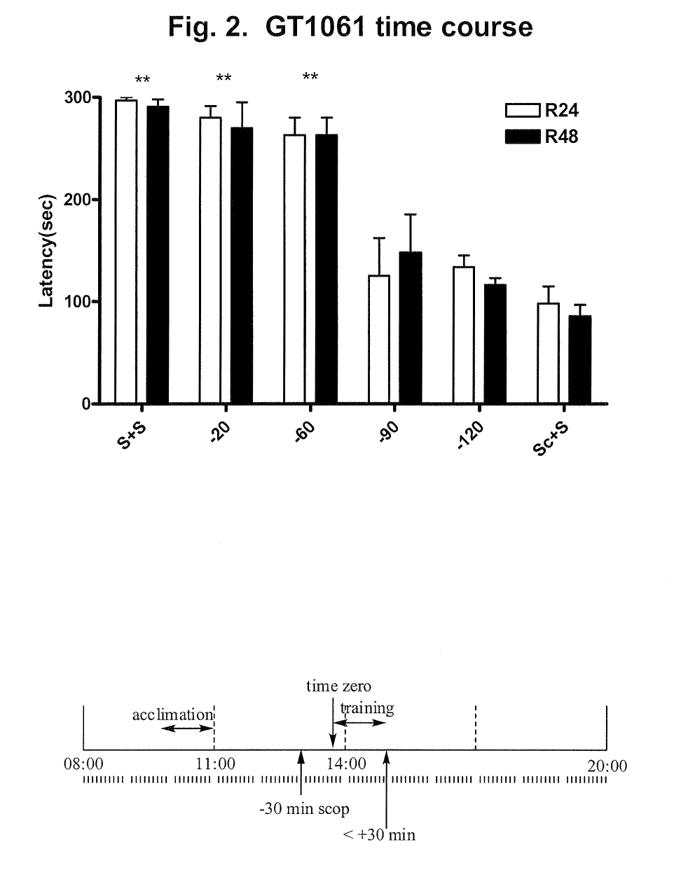

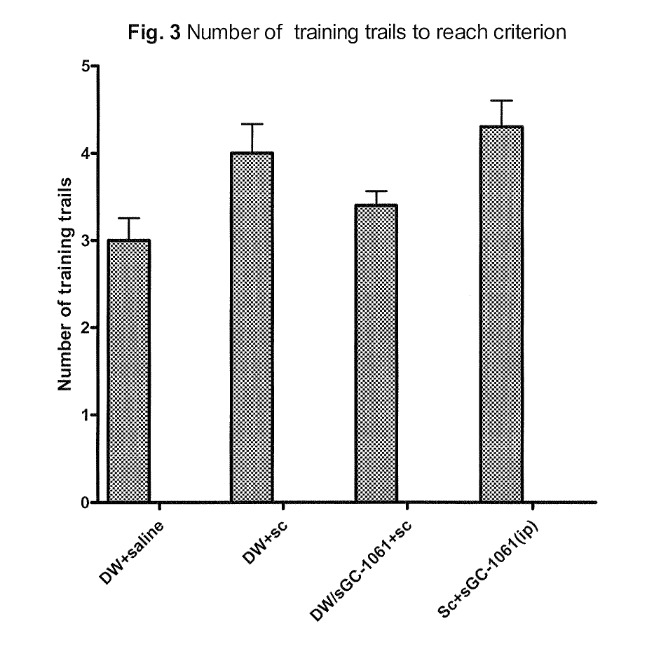

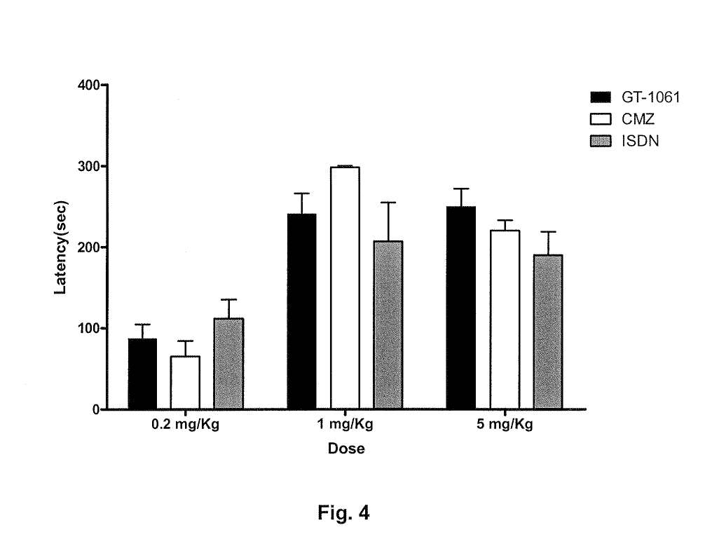

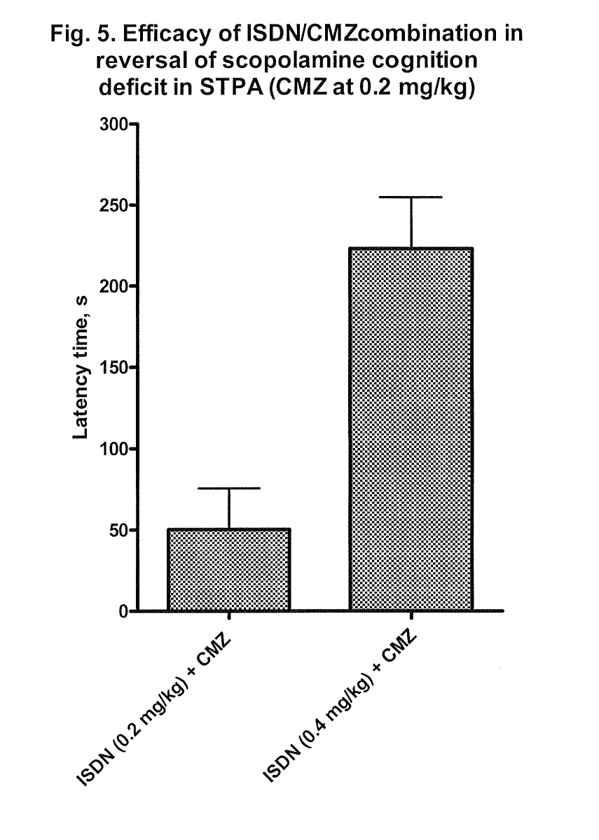

The results from these experiments are shown in FIGS. 1-5.

### EQUIVALENTS

Those skilled in the art will recognize, or be able to ascertain using no more than routine experimentation, many equivalents to the specific embodiments of the invention described herein. Such equivalents are intended to be encompassed by the following claims.

### INCORPORATION BY REFERENCE

The entire contents of all patents published patent applications and other references cited herein are hereby expressly incorporated herein in their entireties by reference.

1. A method for inhibiting neurodegeneration, or effecting neuroprotection in a subject in need thereof, said method comprising administering to said subject ISDN, such that said neurodegeneration is inhibited or said neuroprotection is effected.

2. The method of claim 1, wherein the ISDN is administered in a low dose.

3. The method of claim 2, wherein the low dose does not induce hypotension in a subject.

4. The method of claim 2, wherein the low dose is lower than a dose of ISDN that produces vasodilation in the subject.

5. The method of claim 1, wherein the ISDN is administered in combination with an additional neuroprotective agent.

6. The method of claim 5, wherein the neuroprotective agent is an acetyl cholinesterase inhibitor, or an NMDA receptor antagonist.

7. The method of claim 6, wherein the acetyl cholinesterase inhibitor is tacrine.

8. The method of claim 6, wherein the NMDA receptor antagonist is memantine.

9. The method of claim 5, wherein the neuroprotective agent is clomethiazole.

10. The method of claim 5, wherein the neuroprotective agent is galantamine, rivastigmine, or donepezil.

11. The method of claim 1, wherein the ISDN is administered orally, intravenously, buccally, transdermally or subcutaneously.

12. The method of claim 1, further comprising administering the ISDN in a pharmaceutically acceptable vehicle.

13. A method for inhibiting neurodegeneration, or effecting neuroprotection in a subject in need thereof, said method comprising administering to said subject an effective amount of a therapeutic composition comprising an NO donor, and a neuroprotective agent, such that said neurodegeneration is inhibited or said neuroprotection is effected.

14. The method of claim 13, wherein the NO donor is nitroglycerin (GTN), isosorbide 5-mononitrate (ISMN), isosorbide dinitrate (ISDN), pentaerythritol tetranitrate (PETN), erthrityl tetranitrate (ETN), S,S-dinitrosodithiol (SSDD), [N-[2-(nitroxyethyl)]-3-pyridinecarboxamide (nicorandil), sodium nitroprusside (SNP), or S-nitroso-N-acetylpenicilamine (SNAP).

15. The method of claim 13, wherein the NO donor is ISDN, ISMN, or GTN.

16. The method of claim 5, wherein the neuroprotective agent is
a) selected from the group consisting of insulin-like growth factor-I (IGF-I), insulin growth-like factor-II (IGF-II), transforming growth factor-β1, activin, growth hormone, nerve growth factor, growth hormone binding protein, IGF-binding proteins (especially IGFBP-), basic fibroblast growth factor, acidic fibroblast growth factor, the hst/Kfgk gene product, fibroblast growth factor 3 (FGF-3), fibroblast growth factor 4 (FGF-4), fibroblast growth factor 6 (FGF-6), keratinocyte growth factor, androgen-induced growth factor, int-2, fibroblast growth factor homologous factor-1 (FHF-1), fibroblast growth factor homologous factor-2 (FHF-2), fibroblast growth factor homologous factor-3 (FHF-3), fibroblast growth factor homologous factor-4 (FHF-4), karatinocyte growth factor 2, glial-activating factor, fibroblast growth factor-10 (FGF-10), fibroblast growth factor-16 (FGF-16), ciliary neurotrophic factor, brain derived growth factor, neurotrophin 3, neurotrophin 4, bone morphogenetic protein 2 (BMP-2), glial-cell line derived neurotrophic factor, activity-dependant neurotrophic factor, cytokine leukemia, inhibiting factor, oncostatin M, interleukin, tumor necrosis factor-α (TNF-α), clomethiazole, kynurenic acid, Semax, FK506 (tacrolimus), L-threo-1-pheyl-2-decanoylamino-3-morpholino-1-propanol, andrenocorticotropin-(4-9 analogue (ORG 2766)) and dizolcipine (MK-801), and selegiline; or
b) the neuroprotective agent is a glutamate antagonist selected from the group consisting of NPS1506, GV1505260, MK-801, and GV150526; or
c) the neuroprotective agent is an alpha-amino-3-hydroxy-5-methyl 4-isoxazole proprionic acid (AMPA) antagonist selected from the group consisting of 2,3-dihydroxy-6-nitro-7-sulfamoylbenzo(f)-quinoxaline (NBQX), LY303070 and LY300164; or
d) the neuroprotective agent is an anti-inflammatory agent directed against mucosal vascular addressing cell adhesion molecular 1 (MAdCAM-1) and/or integrin α4 receptors or anti-MAdCAM-1mAb MECA-367 (ATCC accession no. HB-9478); or
e) said neuroprotective agent is a combination of two or more agents provided in a) through d).

17. The method of claim 13, wherein the neuroprotective agent is an acetyl cholinesterase inhibitor, or an NMDA receptor antagonist.

18. The method of claim 17, wherein the acetyl cholinesterase inhibitor is tacrine.

19. The method of claim 17, wherein the NMDA receptor antagonist is memantine.

20. The method of claim 13, wherein the neuroprotective agent is clomethiazole.

21-59. (canceled)
